(19) United States
(12) Patent Application Publication
(10) Pub. No.: US 2008/0183106 A1
(43) Pub. Date: Jul. 31, 2008

(54) INTERACTIVE BABY TIMER SYSTEM AND METHOD

(76) Inventors: Mark D. Covelli, Cave Creek, AZ (US); Christopher V. Mielke, Scottsdale, AZ (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 12/021,691

(22) Filed: Jan. 29, 2008

Related U.S. Application Data
(60) Provisional application No. 60/887,265, filed on Jan. 30, 2007.

(51) Int. Cl. A61B 5/103

(52) U.S. Cl. 600/588

(57) ABSTRACT

Interactive fun, facts and functionality are incorporated into a pregnancy monitoring device. The system includes a contraction monitoring device and timer which are configured to detect contractions to enable a calculation of the length of the contraction and the time between the contractions. The device provides additional audio and visual information related to the pregnancy. The display may include pregnancy status levels such as: emergency level (go to hospital now), prepare for emergency level (get ready to go to hospital), and no emergency level, along with displaying the fetus at certain developmental stages. The audio device may announce special dates and reminders.

### CROSS-REFERENCE TO RELATED APPLICATIONS

This application claims priority to, and the benefits of, U.S. Provisional Application No. 60/887,265 filed on Jan. 30, 2007, the entirety of which is hereby incorporated by reference.

### FIELD OF INVENTION

The present invention generally relates to pregnancy devices, and more particularly, to interactive timers used in association with a pregnancy.

### BACKGROUND OF THE INVENTION

For centuries, pregnant women have struggled with the ability to accurately track the length and period of their contractions. The tracking procedure is very cumbersome and difficult when dealing with the pain of a contraction. The process includes watching the start time of the contraction and then the time when it ends to calculate the length of the contraction. The next step includes timing when the contraction starts again, and subtracting the new start time from the previous start time to determine the time between contractions. Both of these numbers are important when contacting a doctor to let the doctor determine your specific stage of pregnancy.

### SUMMARY OF THE INVENTION

The present invention includes a system and method for providing interactive fun, facts and functionality to a pregnancy monitoring device. The system includes a contraction monitoring device 14 and timer 16 which are configured to detect contractions to enable a calculation of the length of the contraction and the time between the contractions, and the device is further configured to provide additional audio and visual information related to the pregnancy. The display 12 may include pregnancy status levels such as: emergency level (go to hospital now), prepare for emergency level (get ready to go to hospital), and no emergency level.

In one embodiment, a pregnancy monitoring device comprises a microprocessor 10 in communication with a memory 32, a display 12, a contraction monitoring device 14, a timer 16; and a power supply. The microprocessor 10 receives input from the contraction monitoring device 14 and the timer 16 to enable a first calculation of a length of the contraction and a second calculation of a time between multiple of the contractions. The microprocessor 10 determines child data and an appropriate pregnancy status level, based on the first calculation and the second calculation. The display 12 receives input related to the child data and the pregnancy status level from the microprocessor 10 and displays the child data and/or the pregnancy status level.

In certain embodiments, the system also includes certain components contained within a housing, a sensor 24 configured to detect vital signs, an audible device 30 configured to provide sounds, a clock 18, a transmitter to transmit information related to the device to a communication device, memory 32 comprising verbal content or musical content, inputs to activate functions of the device, the microprocessor 10 configured to receive parent data related to a parent and create a display signal related to the parent data and the child data, the microprocessor 10 configured to create a display signal or an audible signal related to advertisements based on the child data, a calendar function for tracking a current date and a special date such that the display 12 displays a statement related to the special date, a representation of a fetus or a child based on the special date, and an audible device 30 to create a sound related to the special date or event.

### BRIEF DESCRIPTION OF THE DRAWINGS

A more complete understanding of the invention may be derived by referring to the detailed description and claims when considered in connection with the Figures, wherein like reference numbers refer to similar elements throughout the Figures, and:

FIG. 1 shows a schematic diagram of the exemplary components of the device, in accordance with various embodiments of the present invention.

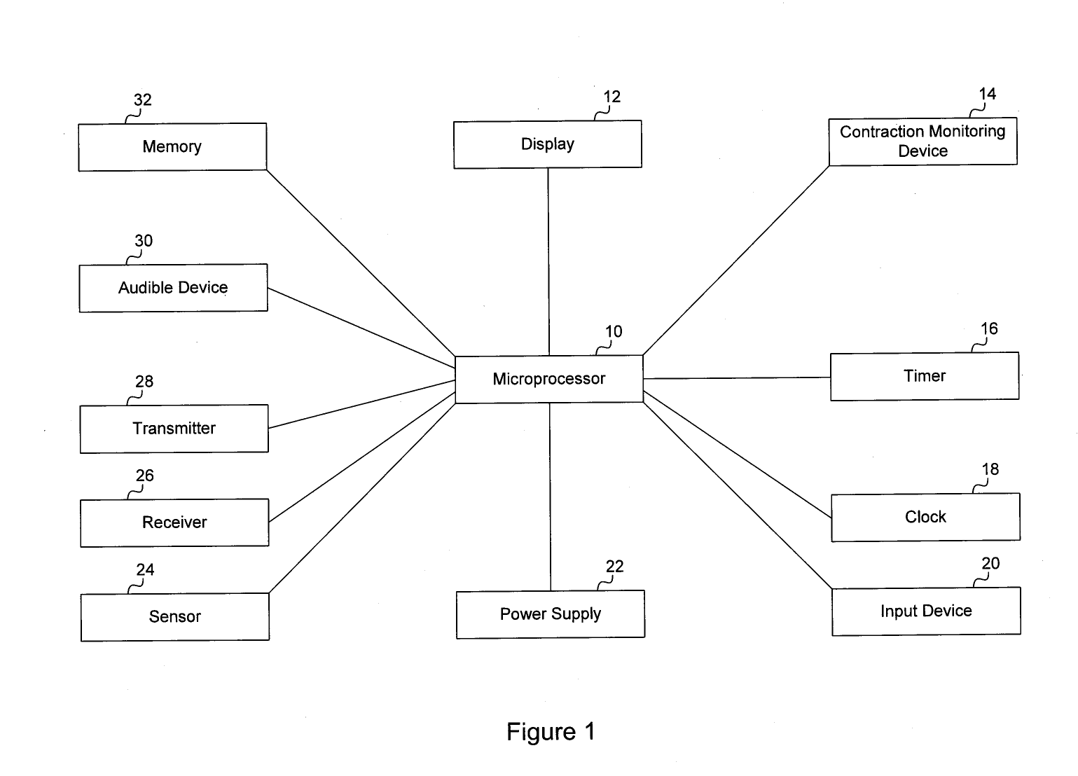

FIG. 2 shows the representative levels displayed by the device, in accordance with various embodiments of the present invention.

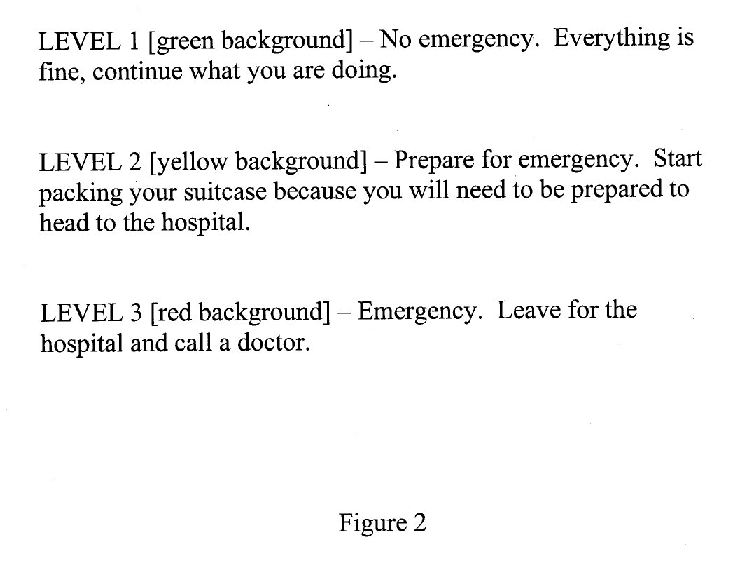

### DETAILED DESCRIPTION

While the representative embodiments are described in sufficient detail to enable those skilled in the art to practice the invention, it should be understood that other embodiments may be realized and that logical and mechanical changes may be made without departing from the spirit and scope of the invention. Thus, the detailed description herein is presented for purposes of illustration only and not of limitation. For example, the steps recited in any of the method or process descriptions may be executed in any order and are not limited to the order presented. Moreover, any of the functions or steps may be outsourced to or performed by one or more third parties. Furthermore, any reference to singular includes plural embodiments, and any reference to more than one component may include a singular embodiment.

For the sake of brevity, conventional data networking, application development and other functional aspects of the systems (and components of the individual operating components of the systems) may not be described in detail herein.

The present invention improves the process and devices related to pregnancy and contraction timing. Specifically, the present invention adds a dimension of interactive fun, facts and functionality to the whole pregnancy process. The system includes a contraction monitoring device 14 and timer 16 which are configured to detect contractions to enable a calculation of the length of the contraction and the time between the contractions. An exemplary contraction monitoring device 14 may include an internal or external device such as, for example, the device disclosed in U.S. Pat. No. 5,070,888, which is hereby incorporated by reference.

In one embodiment of the present invention, the interactive timer is programmed to the expected due date, and periodic (e.g., daily, weekly or monthly) events that are pertinent to a child's development are displayed on the timer 16. More particularly, in one embodiment, the system determines the number of days between a present date and the date of one of the special days that is next to occur in time (e.g., days until next ultrasound, expected date for development of the fingers, days until expected birth date, etc). When said number of days falls within a predetermined range of days, the system audibly or visually communicates a spoken message to alert a mother to the number of days until said one special day arrives.

The device may also be integrated with, and/or get updates from, any other database or system (e.g., Microsoft Outlook, cellphone database, calendaring database, etc) via wired or wireless technology. Any of the data or information discussed herein may be entered directly into the device via any input device 20 known in the art, entered into an email, calendaring system or webpage which sends a signal (containing the information) to the device (e.g. similar to the functionality of a personal digital assistant, Blackberry, etc), transmitted to the device by a sensor 24 and/or the like. While the system may be described as “displaying” certain results, the system also contemplates audible sounds based on certain results, or transmitting such data to other devices. For example, the system may transmit (periodically, on command, at predetermined times, etc) any data or information discussed herein to a computer, a network, cellular phone, MP3 Player, a predetermined distribution list via a text message or email, or any other computing device. The device may include radio technology, infrared technology, cellular technology, RFID technology and/or any other receiving or transmitting technology known in the art or hereinafter developed.

The audio or visual display 12 may be activated by the contraction monitor device, the clock 18, the timer 16, the microprocessor 10 or any other component discussed herein. In accordance with one embodiment of the present invention, audio attributes comprising simulated natural and synthetic sounds are synchronized with the baby's gestation period as it gets closer to delivery time. The audio signals may include messages, which may be specific words, phrases or sentences, different songs, or sound effects such as crying, whining, giggling, chewing, or drinking, which communicate to the mother the baby's simulated actions, thoughts, feelings and emotions, for example.

Coupled with the ability to recognize various inputs from the operator, the timer 16 has the ability for multi-stage interaction. The device may receive input regarding parent data, wherein the parent data may cause the display of features, actions, or characteristics of the father or mother that are starting to develop. In one embodiment, the device may include algorithms for analyzing the father and mother's genes, features or other characteristics to develop the projected combined features or characteristics that the newborn may inherit.

The device may depict a child, adult, animal, cartoon, fantasy figure or any other illustration. The device may also include, for example, a clock 18 for setting the time of day and/or time of year. The system may also include a speech synthesizer, a non-volatile memory 32, a loudspeaker, and/or actuated sensors 24 distributed about the device. The device may also include an internally-carried power supply 22. In other embodiments, the power source may include any power source known in the art such as solar, re-chargeable, mechanical, and the like. The preprogrammed or programmable microprocessor 10 may contain a program which calculates the length and period of the contractions. The processor may cause a command to issue audible or visual signals based on the timer 16 or clock 18 information and relating to pregnancy such as, for example, social (e.g., plan baby shower), religious (e.g., plan christening), financial (e.g., college education savings), administrative (e.g., obtain a passport) or governmental (e.g., obtain a social security number) items that are appropriate to do at a specific time during or after the pregnancy.

The program may also cause the microprocessor 10 to illuminate one of three color LED's or any other audio or visual indicators. Each color correlates to a predetermined level of readiness for the expecting mother. The levels may be determined based upon the contraction information, pre-programmed dates, input from a sensor, or any other input. One level indicates that everything is fine, continue what you are doing. Another level to start packing your suitcase you will need to be prepared to head to the hospital. Another level to leave for the hospital and call a doctor. The device may also display or announce the phone number of your doctor, hospital, parents, friends, etc. The device may also transmit a signal to a cellular phone or other device to automatically contact a doctor or other emergency personnel. In one embodiment, three levels of “intervals” exist. In one embodiment, the three levels include an audible attribute and/or visual display of an unborn fetus. The levels may also include a visual signal flashing in the appropriate colors indicated by the “interval.”

As a further addition, the clock 18 may be of the form of a programmable clock calendar. Birthdays, holidays, local seasons, religious holidays, or religious events and the like may be programmed into the calendar, and the microprocessor 10 program. The repertoire of stored verbal messages may include those which may be correlated to such events or occasions so as to be spoken or displayed by the device on the appropriate day or in anticipation thereof.

The invention includes a system, method, hardware and/or software suitably configured to provide information to an expectant mother, other person or computer regarding the status of various pregnancy related changes or factors. The invention may be implemented in software, hardware, webpages and/or a variety of devices such as cell phones, computer displays and PDAs. In one embodiment, the information generated by the invention may be transmitted to (via transmitter 28) or received from (via receiver 26) any other software and/or hardware via any communication system or network discussed herein. As will be appreciated by one of ordinary skill in the art, the system may be embodied as a customization of an existing system, an add-on product, upgraded software, a stand alone system, a distributed system, a method, a data processing system, a device for data processing, and/or a computer program product. Accordingly, the system may take the form of an entirely software embodiment, an entirely hardware embodiment, or an embodiment combining aspects of both software and hardware. Furthermore, the system may take the form of a computer program product on a computer-readable storage medium having computer-readable program code means embodied in the storage medium. Any suitable computer-readable storage medium may be utilized, including hard disks, CD-ROM, optical storage devices, magnetic storage devices, and/or the like.

In one embodiment, the invention includes an interactive device which contains a clock 18 or other timekeeping device. The timer 16 also has a function that allows the device to count down to a predetermined delivery date. The device also includes one or more displays 12 that would activate by means of a push button.

By initiating the button the first time, the device displays a “start the length of contraction” screen and the “period between contractions”. The button would then be initiated again to stop the “length of contraction” timer 16. The third initiation of the button stops the “period of contraction” timer 16 and restarts the “length of contraction.” This sequence would continue until a pre-determined interval. In one embodiment, the system may automatically detect and/or determine such data without the need for using manual input or button selection. For example, the device may include a strain sensor to automatically detect a contraction, thereby allowing the device to be used while sleeping.

In one embodiment, the outside of the device or the display 12 is used to advertise pregnancy products, baby products, safety products, motherhood products, gift registries, internet websites, doctors, medical device suppliers, pharmaceutical companies and/or the like. In another embodiment, the device provides either daily, weekly or monthly updates on factual information about the baby's development. The updates are displayed based on the programmed expectant delivery date.

The various system components discussed herein may include one or more of the following: a host server or other computing systems including a processor for processing digital data; a memory 32 coupled to the processor for storing digital data; an input digitizer coupled to the processor for inputting digital data; an application program stored in the memory 32 and accessible by the processor for directing processing of digital data by the processor; a display device coupled to the processor and memory for displaying information derived from digital data processed by the processor; and a plurality of databases. Various databases used herein may include: client data; merchant data; financial institution data; and/or like data useful in the operation of the system. As those skilled in the art will appreciate, user computer may include an operating system (e.g., Windows NT, 95/98/2000, XP, Vista, OS2, UNIX, Linux, Solaris, MacOS, etc.) as well as various conventional support software and drivers typically associated with computers. The computer may include any suitable personal computer, network computer, workstation, minicomputer, mainframe or the like. User computer can be in a home or business environment with access to a network. In an exemplary embodiment, access is through a network or the Internet through a commercially-available web-browser software package.

As used herein, the term “network” shall include any electronic communications means which incorporates both hardware and software components of such. Communication among the parties may be accomplished through any suitable communication channels, such as, for example, a telephone network, an extranet, an intranet, Internet, point of interaction device (point of sale device, personal digital assistant (e.g., Palm Pilot®, Blackberry®), cellular phone, kiosk, etc.), online communications, satellite communications, off-line communications, wireless communications, transponder communications, local area network (LAN), wide area network (WAN), networked or linked devices, keyboard, mouse and/or any suitable communication or data input modality. Moreover, although the system is frequently described herein as being implemented with TCP/IP communications protocols, the system may also be implemented using IPX, Appletalk, IP-6, NetBIOS, OSI or any number of existing or future protocols. If the network is in the nature of a public network, such as the Internet, it may be advantageous to presume the network to be insecure and open to eavesdroppers. Specific information related to the protocols, standards, and application software utilized in connection with the Internet is generally known to those skilled in the art and, as such, need not be detailed herein. See, for example, DILIP NAIK, INTERNET STANDARDS AND PROTOCOLS (1998); JAVA 2 COMPLETE, various authors, (Sybex 1999); DEBORAH RAY AND ERIC RAY, MASTERING HTML 4.0 (1997); and LOSHIN, TCP/IP CLEARLY EXPLAINED (1997) and DAVID GOURLEY AND BRIAN TOTTY, HTTP, THE DEFINITIVE GUIDE (2002), the contents of which are hereby incorporated by reference.

The various system components may be independently, separately or collectively suitably coupled to the network via data links which includes, for example, a connection to an Internet Service Provider (ISP) over the local loop as is typically used in connection with standard modem communication, cable modem, Dish networks, ISDN, Digital Subscriber Line (DSL), or various wireless communication methods, see, e.g., GILBERT HELD, UNDERSTANDING DATA COMMUNICATIONS (1996), which is hereby incorporated by reference. It is noted that the network may be implemented as other types of networks, such as an interactive television (ITV) network. Moreover, the system contemplates the use, sale or distribution of any goods, services or information over any network having similar functionality described herein.

As used herein, “transmit” may include sending electronic data from one system component to another over a network connection. Additionally, as used herein, “data” may include encompassing information such as commands, queries, files, data for storage, and the like in digital or any other form.

The system contemplates uses in association with web services, utility computing, pervasive and individualized computing, security and identity solutions, autonomic computing, commodity computing, mobility and wireless solutions, open source, biometrics, grid computing and/or mesh computing.

Any databases discussed herein may include relational, hierarchical, graphical, or object-oriented structure and/or any other database configurations. Common database products that may be used to implement the databases include DB2 by IBM (White Plains, N.Y.), various database products available from Oracle Corporation (Redwood Shores, Calif.), Microsoft Access or Microsoft SQL Server by Microsoft Corporation (Redmond, Wash.), or any other suitable database product. Moreover, the databases may be organized in any suitable manner, for example, as data tables or lookup tables. Each record may be a single file, a series of files, a linked series of data fields or any other data structure. Association of certain data may be accomplished through any desired data association technique such as those known or practiced in the art. For example, the association may be accomplished either manually or automatically. Automatic association techniques may include, for example, a database search, a database merge, GREP, AGREP, SQL, using a key field in the tables to speed searches, sequential searches through all the tables and files, sorting records in the file according to a known order to simplify lookup, and/or the like. The association step may be accomplished by a database merge function, for example, using a “key field” in pre-selected databases or data sectors.

One skilled in the art will also appreciate that, for security reasons, any databases, systems, devices, servers or other components of the system may consist of any combination thereof at a single location or at multiple locations, wherein each database or system includes any of various suitable security features, such as firewalls, access codes, encryption, decryption, compression, decompression, and/or the like.

The computing unit of the web client may be further equipped with an Internet browser connected to the Internet or an intranet using standard dial-up, cable, DSL or any other Internet protocol known in the art. Transactions originating at a web client may pass through a firewall in order to prevent unauthorized access from users of other networks. Further, additional firewalls may be deployed between the varying components of CMS to further enhance security.

Firewall may include any hardware and/or software suitably configured to protect CMS components and/or enterprise computing resources from users of other networks. Further, a firewall may be configured to limit or restrict access to various systems and components behind the firewall for web clients connecting through a web server. Firewall may reside in varying configurations including Stateful Inspection, Proxy based and Packet Filtering among others. Firewall may be integrated within a web server or any other CMS components or may further reside as a separate entity.

The computers discussed herein may provide a suitable website or other Internet-based graphical user interface which is accessible by users. In one embodiment, the Microsoft Internet Information Server (IIS), Microsoft Transaction Server (MTS), and Microsoft SQL Server, are used in conjunction with the Microsoft operating system, Microsoft NT web server software, a Microsoft SQL Server database system, and a Microsoft Commerce Server. Additionally, components such as Access or Microsoft SQL Server, Oracle, Sybase, Informix MySQL, Interbase, etc., may be used to provide an Active Data Object (ADO) compliant database management system.

Any of the communications, inputs, storage, databases or displays discussed herein may be facilitated through a website having web pages. The term “web page” as it is used herein is not meant to limit the type of documents and applications that might be used to interact with the user. For example, a typical website might include, in addition to standard HTML documents, various forms, Java applets, JavaScript, active server pages (ASP), common gateway interface scripts (CGI), extensible markup language (XML), dynamic HTML, cascading style sheets (CSS), helper applications, plug-ins, and the like. A server may include a web service that receives a request from a web server, the request including a URL (http://yahoo.com/stockquotes/ge) and an IP address (123.56.789.234). The web server retrieves the appropriate web pages and sends the data or applications for the web pages to the IP address. Web services are applications that are capable of interacting with other applications over a communications means, such as the internet. Web services are typically based on standards or protocols such as XML, SOAP, WSDL and UDDI. Web services methods are well known in the art, and are covered in many standard texts. See, e.g., ALEX NGHIEM, IT WEB SERVICES: A ROADMAP FOR THE ENTERPRISE (2003), hereby incorporated by reference.

Practitioners will also appreciate that there are a number of methods for displaying data within a browser-based document. Data may be represented as standard text or within a fixed list, scrollable list, drop-down list, editable text field, fixed text field, pop-up window, and the like. Likewise, there are a number of methods available for modifying data in a web page such as, for example, free text entry using a keyboard, selection of menu items, check boxes, option boxes, and the like.

The system and method may be described herein in terms of functional block components, screen shots, optional selections and various processing steps. It should be appreciated that such functional blocks may be realized by any number of hardware and/or software components configured to perform the specified functions. For example, the system may employ various integrated circuit components, e.g., memory elements, processing elements, logic elements, look-up tables, and the like, which may carry out a variety of functions under the control of one or more microprocessors or other control devices. Similarly, the software elements of the system may be implemented with any programming or scripting language such as C, C++, Macromedia Cold Fusion, Microsoft Active Server Pages, Java, COBOL, assembler, PERL, Visual Basic, SQL Stored Procedures, extensible markup language (XML), with the various algorithms being implemented with any combination of data structures, objects, processes, routines or other programming elements. Further, it should be noted that the system may employ any number of conventional techniques for data transmission, signaling, data processing, network control, and the like. Still further, the system could be used to detect or prevent security issues with a client-side scripting language, such as JavaScript, VBScript or the like. For a basic introduction of cryptography and network security, see any of the following references: (1) “Applied Cryptography: Protocols, Algorithms, And Source Code In C,” by Bruce Schneier, published by John Wiley & Sons (second edition, 1995); (2) “Java Cryptography” by Jonathan Knudson, published by O'Reilly & Associates (1998); (3) “Cryptography & Network Security: Principles & Practice” by William Stallings, published by Prentice Hall; all of which are hereby incorporated by reference.

These computer program instructions may be loaded onto a general purpose computer, special purpose computer, or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

Accordingly, functional blocks of the block diagrams and flowchart illustrations support combinations of means for performing the specified functions, combinations of steps for performing the specified functions, and program instruction means for performing the specified functions. It will also be understood that each functional block of the block diagrams and flowchart illustrations, and combinations of functional blocks in the block diagrams and flowchart illustrations, can be implemented by either special purpose hardware-based based computer systems which perform the specified functions or steps, or suitable combinations of special purpose hardware and computer instructions. Further, illustrations of the process flows and the descriptions thereof may make reference to user windows, webpages, websites, web forms, prompts, etc. Practitioners will appreciate that the illustrated steps described herein may comprise in any number of configurations including the use of windows, webpages, web forms, popup windows, prompts and the like. It should be further appreciated that the multiple steps as illustrated and described may be combined into single webpages and/or windows but have been expanded for the sake of simplicity. In other cases, steps illustrated and described as single process steps may be separated into multiple webpages and/or windows but have been combined for simplicity.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the invention. The scope of the invention is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. All structural, chemical, and functional equivalents to the elements of the above-described exemplary embodiments that are known to those of ordinary skill in the art are expressly incorporated herein by reference and are intended to be encompassed by the present claims. Further, a list of elements does not include only those elements but may include other elements not expressly listed or inherent to such process, method, article, or apparatus.

1. A pregnancy monitoring device comprising:
a microprocessor in communication with a memory;
a display in communication with said microprocessor;
a contraction monitoring device in communication with said microprocessor and configured to detect a contraction;
a timer in communication with said microprocessor;
a power supply coupled to said microprocessor;
said microprocessor configured to receive input from said contraction monitoring device and said timer to enable a first calculation of a length of said contraction and a second calculation of a time period between multiple of said contractions;
said microprocessor configured to determine child data and an appropriate pregnancy status level, based on said first calculation and said second calculation;
said display configured to receive input related to at least one of: said child data and said pregnancy status level from said microprocessor; and,
said display configured to display at least one of child data and said pregnancy status level.

2. The device of claim 1, wherein said microprocessor, display, contraction monitoring device, timer and power supply are contained within a housing.

3. The device of claim 1, further comprising a sensor in communication with said microprocessor and configured to detect vital signs.

4. The device of claim 1, further comprising an audible device in communication with said microprocessor and configured to provide sounds.

5. The device of claim 1, further comprising a clock in communication with said contraction monitoring device.

6. The device of claim 1, further comprising at least one of a transmitter to transmit information related to said device to a communication device and a receiver to receive information from at least one of a database and second device.

7. The device of claim 1, wherein said memory comprises at least one of: verbal content and musical content.

8. The device of claim 1, further comprising an input to activate functions of said device related to said contraction monitoring device.

9. The device of claim 1, wherein said microprocessor is configured to receive parent data related to a parent and create a display signal related to said parent data and said child data.

10. The device of claim 1, wherein said microprocessor is configured to create at least one of: a display signal and an audible signal related to advertisements based on said child data.

11. The device of claim 1, further comprising a calendar function for tracking a current date and a special date.

12. The device of claim 1, further comprising a calendar function for tracking a current date and a special date, wherein at least one of said display is further configured to display a statement related to said special date and said audible device is further configured to create a sound related to said special date.

13. The device of claim 1, further comprising a calendar function for tracking a current date, a special date and a special event, said display configured to display a representation of at least one of a fetus and a child based on said special date.

14. The device of claim 1, wherein said pregnancy status level includes at least one of: emergency level, prepare for emergency level, and no emergency level.

15. The device of claim 1, wherein said microprocessor further comprises a verbal statement program for assembling a selected verbal statement from words and phrases stored in said memory.

16. A method for displaying an indicator on a pregnancy monitoring device, said method comprising:
receiving, at a processor, input from a contraction monitoring device and a timer to enable a first calculation of a length of said contraction and a second calculation of a time between multiple of said contractions;
determining child data and an appropriate pregnancy status level, based on said first calculation and said second calculation; and,
displaying at least one of said child data and said pregnancy status level on said pregnancy monitoring device, wherein said pregnancy monitoring device comprises a microprocessor in communication with a memory, a display in communication with said microprocessor, said contraction monitoring device in communication with said microprocessor, a timer in communication with said microprocessor, and a power supply coupled to said microprocessor.
